(19) United States
(12) Patent Application Publication
(10) Pub. No.: US 2026/0107989 A1
(43) Pub. Date: Apr. 23, 2026

(54) ADJUSTABLE POST-OPERATIVE RECOVERY GARMENT

(71) Applicant: Design With Purpose LLC, Columbus, OH (US)

(72) Inventor: Kristen Neumeier, Columbus, OH (US)

(21) Appl. No.: 19/315,203

(22) Filed: Aug. 29, 2025

Related U.S. Application Data
(60) Provisional application No. 63/689,568, filed on Aug. 30, 2024.

(51) Int. Cl. A41C 3/00; A61M 1/00; A61M 27/00

(52) U.S. Cl. CPC A41C 3/0064; A61M 1/682; A61M 1/84; A61M 27/00; A61M 2210/1007

(57) ABSTRACT

A garment for post-surgical recovery includes a torso covering body formed from a first fabric layer and a second fabric layer bonded together by a thermoplastic polyurethane layer. The thermoplastic polyurethane bonding extends between the fabric layers along contours of the torso covering body to provide a stable layered construction without rigid inserts. The garment may include shoulder straps extending upwardly from the torso covering body and an opening region disposed along either a front or rear central portion. In some embodiments a vertical seam defines a front opening region, and in other embodiments a fastening mechanism defines a rear opening region. The fastening mechanism may include a securing element positioned to maintain closure of the garment. The bonded multilayer construction may be configured to provide extended wear and structural stability for recovery following surgical procedures involving the torso.

### CROSS REFERENCE TO RELATED APPLICATION

This Application claims priority to U.S. Provisional Application No. 63/689,568, titled “ADJUSTABLE POST-OPERATIVE RECOVERY GARMENT,” filed Aug. 30, 2024, the entire disclosure of which is hereby incorporated by reference for all purposes.

### TECHNICAL FIELD

Embodiments described herein relate to clothing articles and, in particular, to chest, shoulder, and back surgery recovery garments that improve comfort to accelerate recovery time.

### BACKGROUND

Post-operative care recommendations for shoulder surgeries, upper back surgeries, chest surgeries, biopsy procedures, lumpectomy procedures, mastectomy procedures, gender-affirming procedures, and other oncological and non-oncological (e.g., cosmetic) chest, back, and axillary reconstruction, reduction, or augmentation procedures varies from patient to patient. In many cases, recommendations include physical therapy because many clinical trials, metanalyses, and other epidemiological studies have demonstrated a positive correlation between physical activity and recovery results.

To assist with recovery, patients are conventionally issued or instructed to purchase general purpose post-surgery support appliances and/or garments. However, these conventional appliances typically do not provide comfortable or customized support and are either too compressive, too loose, or otherwise are improperly fit and motivate patients to modify, cut, discard, or decline to wear them, delaying recovery times, increasing chances for post-surgery injury, risking infections resulting from fluid build-up, and extending overall discomfort.

Furthermore, due to these and other causes of post-surgical pain and discomfort, many surgery patients avoid or ignore physical therapy recommendations, which may further delay recovery, potentially result in a permanent post-surgical physical decline and/or chronic pain.

Further still, many post-surgical patients experience disturbed or disordered sleep, which may also delay recovery and increase the likelihood of negative outcomes. Patient experiences of protracted discomfort, poor sleep, and suboptimal recovery can contribute to acute or chronic declines in mental health. These very negative patient experiences can have further negative collateral effects when these patients recount their experiences to friends, family, or medical practitioners, having the effect of potentially discouraging others from undergoing and/or recommending medically necessary procedures.

### BRIEF DESCRIPTION OF THE DRAWINGS

Reference will now be made to representative embodiments illustrated in the accompanying figures. It should be understood that the following descriptions are not intended to limit this disclosure to one included embodiment. To the contrary, the disclosure provided herein is intended to cover alternatives, modifications, and equivalents as may be included within the spirit and scope of the described embodiments, and as defined by the appended claims.

FIG. 1 depicts an example isometric view of a post-surgery support garment as described herein.

FIG. 2 depicts a front exterior plan view of the post-surgery support garment as depicted in FIG. 1.

FIG. 3 depicts a first side exterior plan view of the post-surgery support garment of FIG. 1.

FIG. 4 depicts a second side exterior plan view of the post-surgery support garment of FIG. 1.

FIG. 5 depicts a back exterior plan view of the post-surgery support garment of FIG. 1.

FIG. 6 depicts a front interior plan view of the post-surgery support garment of FIG. 1.

FIG. 7 depicts a side interior plan view of the post-surgery support garment of FIG. 1.

FIG. 8 depicts a back interior plan view of the post-surgery support garment of FIG. 1.

FIGS. 9-10 depict the post-surgery support garment of FIG. 1 with seams that define cup structure.

FIG. 11 depicts an example embodiment of the post-surgery support garment of FIG. 1 in which a closure tab may be trimmable by a patient.

FIGS. 12-14 depict an construction of a post-surgery support garment such as shown in FIG. 1 with protected seams, configurable straps, and bonded multilayer construction.

The use of the same or similar reference numerals in different figures indicates similar, related, or identical items.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Embodiments described herein relate to garments that provide support and comfort to a post-operative breast surgery patient, thereby enabling the patient to engage in recovery-promoting and recovery-accelerating activities, such as physical therapy or uninterrupted sleep. For simplicity of description and illustration the embodiments that follow reference a bra, but it may be appreciated that other garments and garment types can be adapted or modified in view of the disclosure provided herein. Such example garments include shirts, girdles, tank-tops, tube-tops, corsets, trainers, slings, compression straps, surgical garments, and the like.

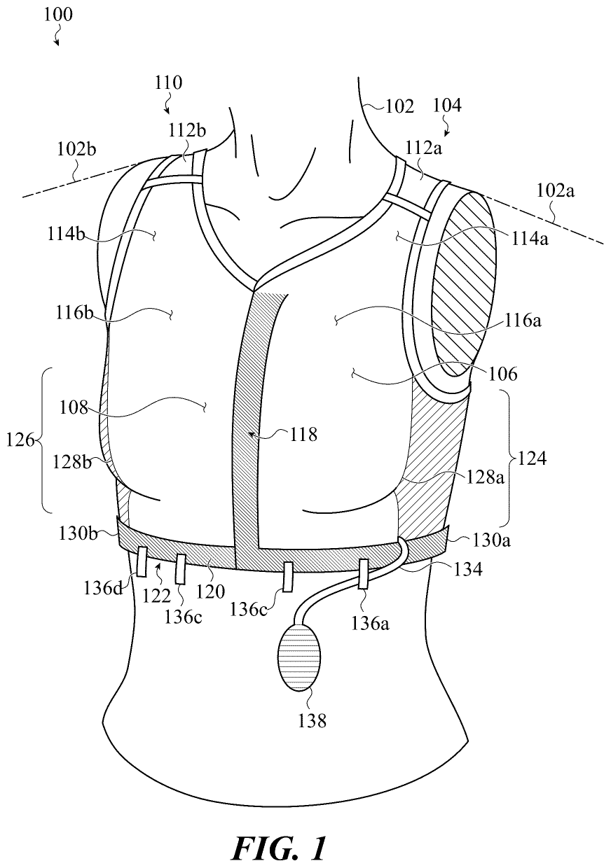

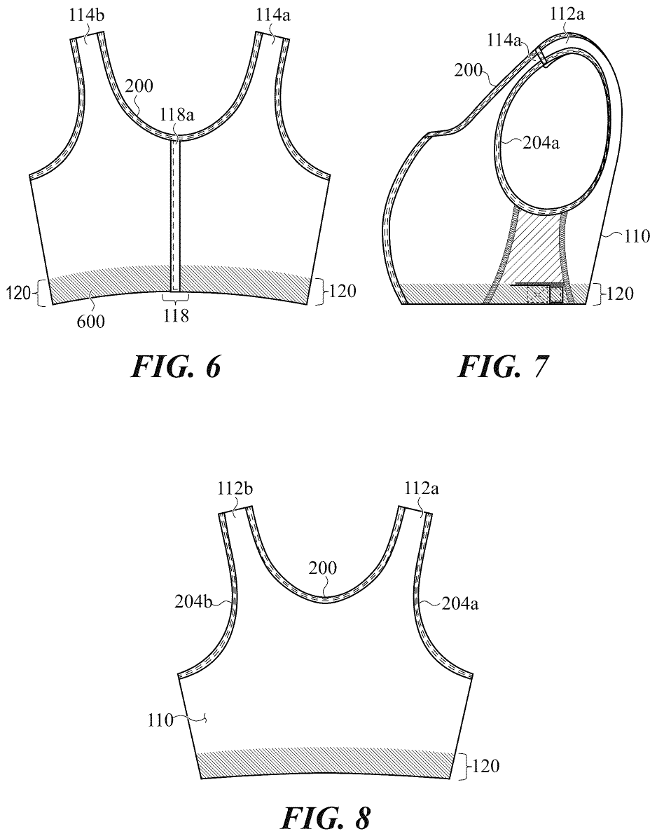

These foregoing and other embodiments are discussed below with reference to FIGS. 1-14. However, those skilled in the art will readily appreciate that the detailed description given herein with respect to these figures is for explanation only and should not be construed as limiting.

Generally and broadly, FIGS. 1-14 depict a post-surgery support garment 100. The post-surgery support garment 100 can be worn by a patient 102 having completed a breast surgery such as, but not limited to, a biopsy procedure, a lumpectomy procedure, a mastectomy procedure, a gender-affirming procedure, or another oncological and non-oncological (e.g., cosmetic) breast reconstruction, reduction, or augmentation.

The post-surgery support garment 100 can be implemented as a bra 104 worn on a chest of the patient 102. The bra 104 can provide compression in certain regions while providing relaxed support in other regions. The bra 104 includes multiple adjustment regions so that the patient 102 can manually adjust fit while wearing the bra 104. Specifically the bra 104 can be particularly structured such that the patient 102 can adjust the fit of the bra 104 along the sternum, along the shoulder line, and along the ribcage to provide multiple degrees of freedom and adjustment.

The bra 104 can include a first front panel 106 and a second front panel 108 configured to cover and support the left side of the chest of the patient 102 and a right side of the chest of the patient 102.

The first front panel 106 can be formed from a multi-way stretch material, such as a four way stretch material. In some cases, the first front panel 106 is formed from a blend of synthetic fibers, such as 90% nylon and 10% elastane. In other cases, the first front panel 106 can be formed from a different ratio of synthetic materials, such as 80% nylon and 20% elastane. In one embodiment, 92% nylon and 8% elastane may be used. In some embodiments, organic materials (e.g., cotton, bamboo, recycled fibers, wool, and so on) of one or more types may also be incorporated. For certain embodiments, a blend of organic and synthetic fibers may be suitable.

The first front panel 106 can be constructed from a single layer of fabric or multiple layers of fabric. For multilayer embodiments, individual layers may be bonded, sewn, or otherwise stitched together to form a single structure.

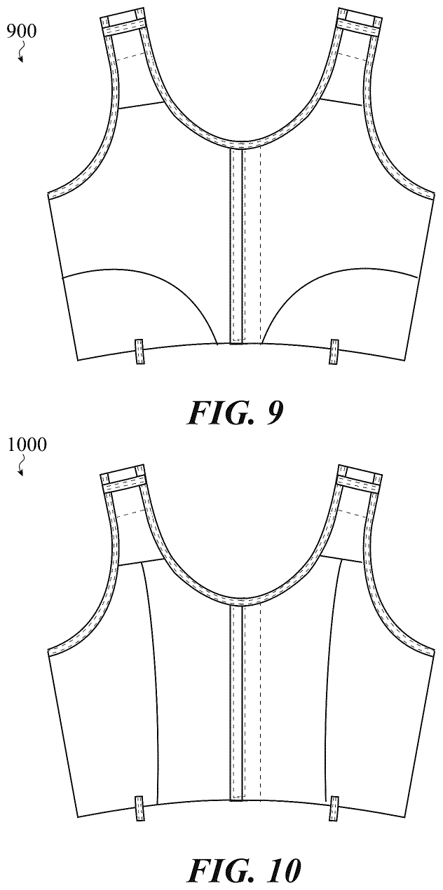

The first front panel 106 can include one or more pockets, channels, or cavities to receive structural inserts, such as padding, boning, or rigid structural inserts. In some cases, the first front panel 106 can be defined by an upper cup portion, a lower cup portion, an inner cup portion, and/or an outer cup portion. In other cases, the first front panel 106 and the second front panel 108 can be molded to adopt a particular support shape. Different cup portions can be formed form different materials sewn or otherwise adhered to one another to define a nonplanar shape configured to contour at least partially to a breast of the patient 102. For example, FIG. 9 depicts an example construction in which a cup can be defined by two seams 900 and 902, bonding multiple sections of the first front panel 106 together to define an upper and lower cup. In another embodiment such as shown in FIG. 10, seams 1000 and 10002 can bond multiple parts of the first front panel 106 and the second front panel 108 to define a left side and right side cup. Many constructions are possible.

Returning to FIGS. 1-8, the first front panel 106 can be woven with multiple elasticities and/or rigidities in different regions of the first front panel 106. For example, an inner cup region near the sternum of the patient 102 can be woven to exhibit less flexibility than an upper cup portion of the first front panel 106.

The second front panel 108 can be configured in the same manner and/or a different manner from the first front panel 106. In many cases, the first front panel 106 and the second front panel 108 are formed form the same material. In other cases, the first front panel 106 and the second front panel 108 are formed from a different material or a different set of materials.

In many cases, the second front panel 108 can be constructed to support the same cup shape as the first front panel 106, whereas in other cases, the second front panel 108 supports a different cup shape than the first front panel 106. For example, for mastectomy patients, the second front panel 108 may be configured to contour to a ribcage of the patient 102 while the first front panel 106 is configured to contour to a breast of the 102.

Further to the foregoing, it may be appreciated that like the first front panel 106, the second front panel 108 can be formed from a multi-way stretch material, such as a four way stretch material. In some cases, as with other fabric sections and/or garment sections as described herein, the second front panel 108 can be formed from a blend of synthetic fibers, such as 90% nylon (e.g., polyamide) and 10% elastane. In other cases, the second front panel 108 can be formed from a different ratio of synthetic materials, such as 80% nylon and 20% elastane. In one embodiment, 92% nylon and 8% elastane may be used. In some embodiments, either or both the first front panel 106 and the second front panel 108 can be formed at least in part from organic fibers and/or a combination of organic and synthetic fibers. It may be appreciated that different breathability, elasticity, support, heat transfer, and other properties may vary by material combination.

The material of the first front panel 106 can be any suitable material weight. In some cases, a weight of less than 100 grams per square meter (“GSM”) may be used. For example, 80 GSM may be used in some cases. In other embodiments, 85 GSM may be used. In yet other cases, 90 GSM may be appropriate. Embodiments and configurations may vary. In other cases, 190 GSM or 300 GSM or higher may be suitable.

A net GSM may be achieved by layering or stacking lighter materials together. For example, two layers of 90 GSM material may result in a patient experience of 180 GSM. Many embodiment and constructions are possible.

As with the first front panel 106, the second front panel 108 can be constructed from a single layer of fabric or multiple layers of fabric. For multilayer embodiments, individual layers may be bonded, sewn, or otherwise stitched together to form a single structure.

The second front panel 108 can include one or more pockets, channels, or cavities to receive structural inserts, such as padding, boning, or rigid structural inserts. In some cases, the second front panel 108 can be defined by an upper cup portion, a lower cup portion, an inner cup portion, and/or an outer cup portion. Different cup portions can be formed form different materials sewn or otherwise adhered to one another to define a nonplanar shape configured to contour at least partially to a breast of the patient 102.

In some cases, the bra 104 and in particular the first front panel 106 and/or second front panel 108 can be configured with one or more holes, apertures or mesh sections to promote particular properties in particular locations for a particular patient. For example, some patients may require or prefer more breathability (e.g., a mesh weave) in a center gore region or yoke region of the bra 104, while other patients may prefer heat retention along the center gore. By varying fabric section, weave density, or other properties of the materials of the first front panel 106 or the second front panel 108, different comfort objectives of different patients can be achieved.

More broadly, as with other sections of the post-surgery support garment 100, the second front panel 108 can be knit with multiple elasticities and/or rigidities in different regions of the first front panel 106. For example, an inner cup region near the sternum of the patient 102 can be knit to exhibit less flexibility than an upper cup portion of the first front panel 106. In many cases, an apex region of the first front panel 106 and/or the second front panel 108 can be configured to exhibit less flexibility than other regions of the bra 104. In some cases, a cradle portion of the bra 104 can be configured to exhibit anisotropic elasticity—around a circumference of the lower ribcage of the patient 102 the cradle may be configured to exhibit a greater elasticity than in a stretch direction perpendicular to that circumference. Many configurations and constructions are possible.

In some cases, the first front panel 106 and/or the second front panel 108 can be integrally formed with a back panel 110. As with other panels described herein, the back panel 110 can be formed from a multi-way stretch material, such as a four way stretch material. In some cases, the back panel 110 may be formed from a blend of synthetic fibers, such as 90% nylon and 10% elastane. In other cases, the back panel 110 can be formed from a different ratio of synthetic materials, such as 80% nylon and 20% elastane. In one embodiment, 92% nylon and 8% elastane may be used. In other embodiments, the back panel 110 can include synthetic and organic fibers. In some cases, the back panel 110 can be formed from organic fibers only.

The back panel 110 can be constructed from a single layer of fabric or multiple layers of fabric. For multilayer embodiments, individual layers may be bonded, sewn, or otherwise stitched together to form a single structure. The back panel 110 can include one or more pockets, channels, or cavities to receive structural inserts, such as padding, boning, or rigid structural inserts. In some cases, the back panel 110 (and/or the first front panel 106 or the second front panel 108) can include one or more thermally conductive inserts, such as heat pipes, fluid channels (with or without a mechanical or manual pump) or thermally conductive materials (e.g., copper threading, thermally conductive plastics, and so on).

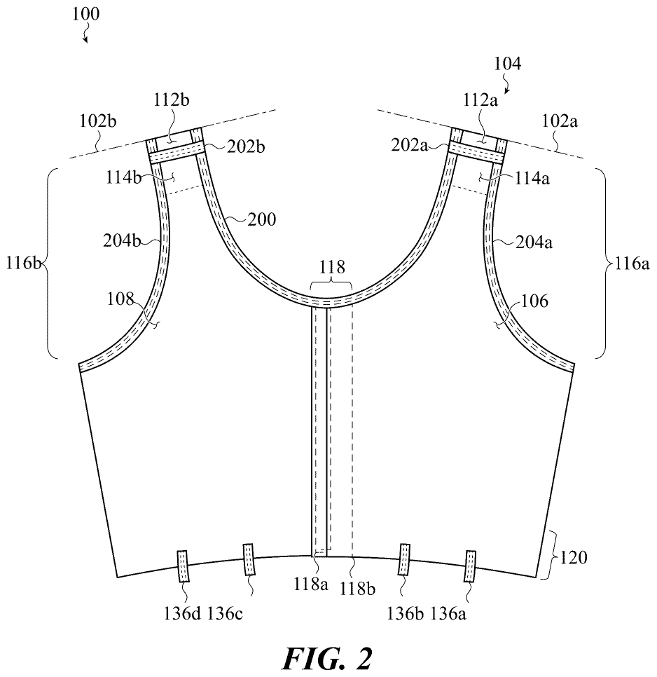

Although depicted in FIG. 2 (and others) as a single contiguous material, in some cases, the back panel 110 can include one or more cutouts, apertures, or openings to facilitate local changes in elasticity, flexibility, rigidity, and/or heat transfer or moisture reduction. More broadly, the back panel 110 can be woven with multiple elasticities and/or rigidities in different regions of the first front panel 106 or the second front panel 108.

The back panel 110 can include two strap sections extending from a body of the back panel 110. The strap sections can be sized and configured to overlap a shoulder line 102a of the patient 102 by a distance d, which may vary by sizing and/or preferences of the patient 102.

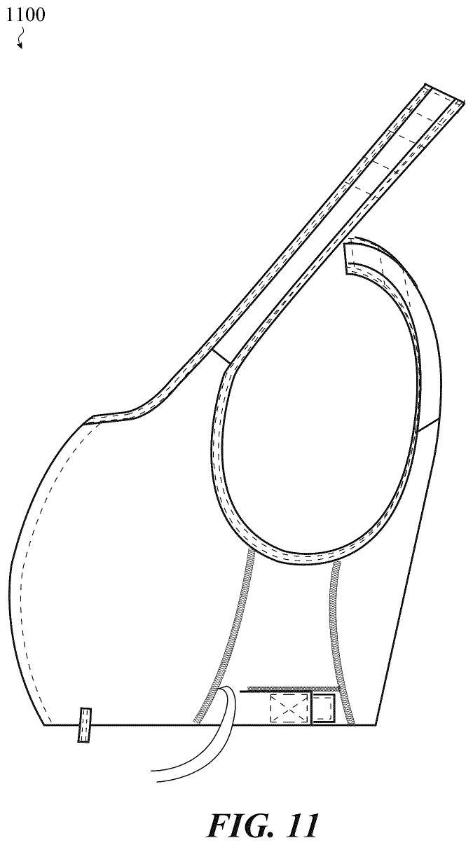

More specifically, the back panel 110 defines an adjustable shoulder strap portion 112a and an adjustable shoulder strap portion 112b that are sized to overlap the shoulder line 102a of the patient 102. In some cases, the adjustable shoulder strap portion 112a and/or the adjustable shoulder strap portion 114a can be extended (see, e.g., FIG. 11) with rows of stitching defining separable segments of the respective strap. As a result of this construction, the patient 102 can select an appropriate length and cut excess of the respective strap away without risking a fraying of the fabric of the first front panel 106 or the back panel 110 (and specifically the adjustable shoulder strap portion 112a or the adjustable shoulder strap portion 114a). It may be appreciated that although FIG. 11 depicts a single strap being extended, any strap or closure as described herein can be extended and sewn in this manner, allowing for patient-specific size customization of side closures, front closures, and shoulder straps.

As a result of this construction, the patient 102 may be able to put on the bra 104 without requiring backward mobility of the arms of the patient 102, which may be restricted and or painful post-surgery. More simply, the patient 102 may be able to wear the bra 104 in much the same manner as a front-closure shirt or jacket, not requiring any manipulation of fabric or closure behind the back of the patient 102.

The adjustable shoulder strap portion 112a and the adjustable shoulder strap portion 112b are configured to engage with an adjustable shoulder strap portion 114a of the first front panel 106 and an adjustable shoulder strap portion 114b of the first front panel 106, respectively. In many embodiments, the adjustable shoulder strap portion 112a and the adjustable shoulder strap portion 114a include complementary closure parts, such as complementary magnets, complementary buttons and buttonholes, complementary snaps and snap bodies, hooks and loops, or the like. For simplicity of description, the embodiments that follow reference hook and loop closures, but it may be appreciated this is merely one example.

The adjustable shoulder strap portion 114a can include a loop portion of a hook and loop closure specifically so that any exposed portion of the adjustable shoulder strap portion 114a does not engage with or snag clothing that may be worn by the patient 102 over the bra 104. Similarly, the adjustable shoulder strap portion 114b can include a loop portion of a hook and loop closure specifically so that any exposed portion of the adjustable shoulder strap portion 114b does not engage with or snag clothing that may be worn by the patient 102 over the bra 104.

The adjustable shoulder strap portion 114a is defined form a first upper front panel portion 116a, which is an upper portion of the first front panel 106. Similarly, the adjustable shoulder strap portion 114b is defined from the second upper front panel portion 116b, which is an upper portion of the second front panel 108.

The first front panel 106 and the second front panel 108 can be configured to engage with one another at a closure front closure 118. The front closure 118 can be implemented as a hook and loop, or another closure mechanism or combination of closure mechanisms. In some cases, a first portion of the front closure 118 is thinner than a second portion of the front closure 118, so that a wider range of circumferential adjustment may be achieved. For example, with reference to FIG. 2, a first front closure portion 118a may be differently sized than a second front closure portion 118b.

As with other closures described herein, the front closure 118 can orient a hook strip of a hook and loop strip such that hooks, which are likely to snag clothing, are oriented inwardly toward the body of the patient 102. Additional material can be provided below the front closure 118 (e.g., an extension portion of the first front panel 106 or the second front panel 108) to prevent a hook from irritating skin of the patient 102.

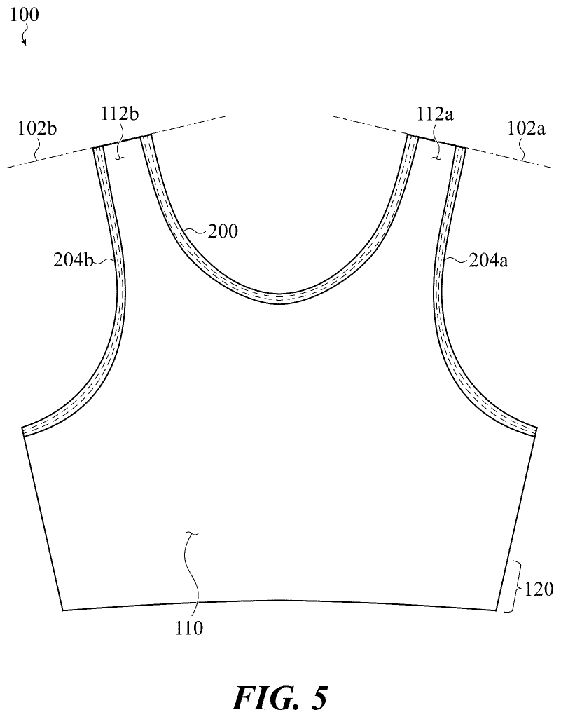

The bra 104 also includes a bottom circumference band 120, which may be elasticated and may include a high-friction/grip coating or bead along a skin-facing surface thereof. As shown in FIG. 1, this section may be referred to as the position retention lining 122. With reference to FIG. 5, this portion may be referred to as the position retention lining 600. The position retention lining 122 or 600 can be formed form an adhesive, elastic, and/or polypropylene based material such as TPU (thermoplastic polyurethane) or TPE (thermoplastic elastomers). For example, in some embodiments, a flexible adhesive can be disposed between two layers of fabric. In other cases, a high friction coating can be disposed on one or more surfaces of a single-ply or multi-ply material. Many constructions are possible.

As noted above, in some embodiments the first front panel 106 may be integrally formed with the back panel 110 and the second front panel 108. In many embodiments, however, a flexible mesh interposes the first front panel 106 and the back panel 110 and likewise the second front panel 108 and the back panel 110.

More specifically, in some embodiments, the bra 104 can include a first side panel 124 and a second side panel 126, which may be formed from a different material than then first front panel 106, the second front panel 108, and the back panel 110. For example, in some cases, the first side panel 124 and the second side panel 126 may be formed from a synthetic blend of 88% polyester and 12% elastane. In other cases, a different ratio may be used. The first side panel 124 and the second side panel 126 can be woven to support or encourage or promote breathability.

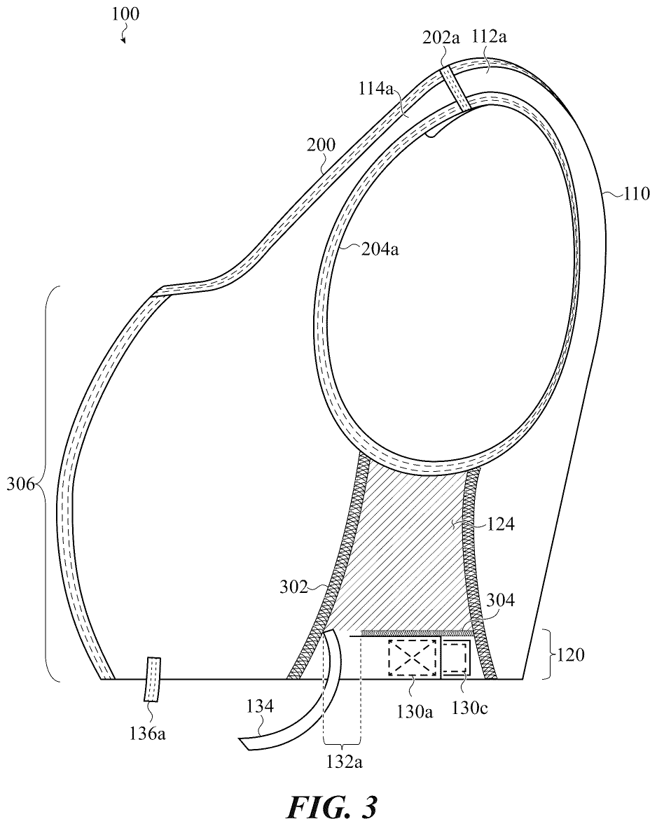

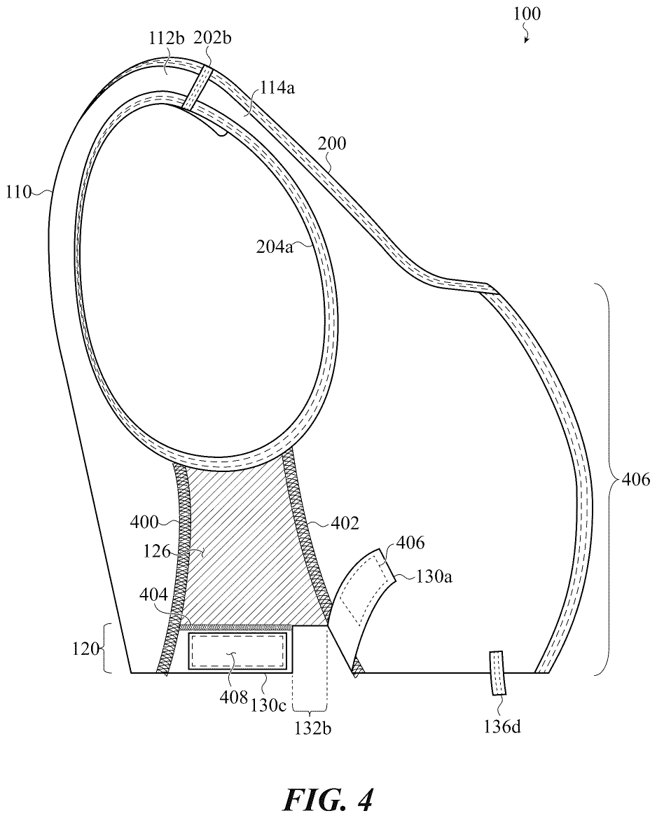

The first side panel 124 can be attached to the first front panel 106 by a first seam 128a (e.g., FIG. 1; see element seam 302 of FIG. 3). Similarly, the second side panel 126 can be attached to the second front panel 108 via a second seam 128b (e.g., FIG. 1; see element seam 402 of FIG. 4).

The first front panel 106 includes an extension tab configured to engage with a corresponding tab extending from the back panel 110 so as to provide adjustability along the ribcage of the patient 102. Specifically, the first front panel 106 includes a first side adjustment strap portion 130a and the second front panel 108 includes a second side adjustment strap portion 130b.

The first side adjustment strap portion 130a is configured to engage with a corresponding tab or portion of the back panel 110. Specifically, the first side adjustment strap portion 130a can engage with and/or define a closure with a first side adjustment strap base 130c (see, e.g., FIG. 3). Likewise, the second side adjustment strap portion 130b can be configured to engage with a second side adjustment strap base 130d.

The first side panel 124 can be attached at least partially to the first side adjustment strap base 130c via a seam 304 (see, FIG. 3). The seam 304 may extend only for a portion of the width of the first side panel 124 to define an aperture through which a drainage tube such as a Jackson-Pratt drainage tube can be fed. For example, the seam 304 can be configured to define a drainage tube access port 132a (see FIGS. 1 and 3). Similarly structure can be symmetrically defined along an opposite side of the bra 104. For example, bra 104 can include a seam 404 that couples the first side adjustment strap base 130c to the back panel 110, defining a drainage tube access port 132b. As a result of this construction a first side adjustment strap portion 130b can be configured to align a first closure part 408 with a second closure part 410 so as to customize an elasticity/flexibility of the second side panel 126.

As a result of this construction, a surgical suction drainage tube 134 can be fed through the drainage tube access port 132a and connected to a surgical suction drainage bulb 138. The surgical suction drainage tube 134 can be secured via one or more drainage tube support straps, such as the drainage tube support strap 136a, t eh drainage tube support strap 136b, the drainage tube support strap 136c, or the drainage tube support strap 136d, which may be a loop of material of a suitable closure type with the bottom circumference band 120 (e.g., hook and loop). As with other embodiments described herein, hooks of the hook and loop closure may be oriented inwardly so as to prevent snagging on clothes.

The bra 104 can also include edge seams, such as the neckline edge seam 200, the first strap edge finishing 202a, the second strap edge finishing 202b, the underarm seam 204a, and the underarm seam 204b.

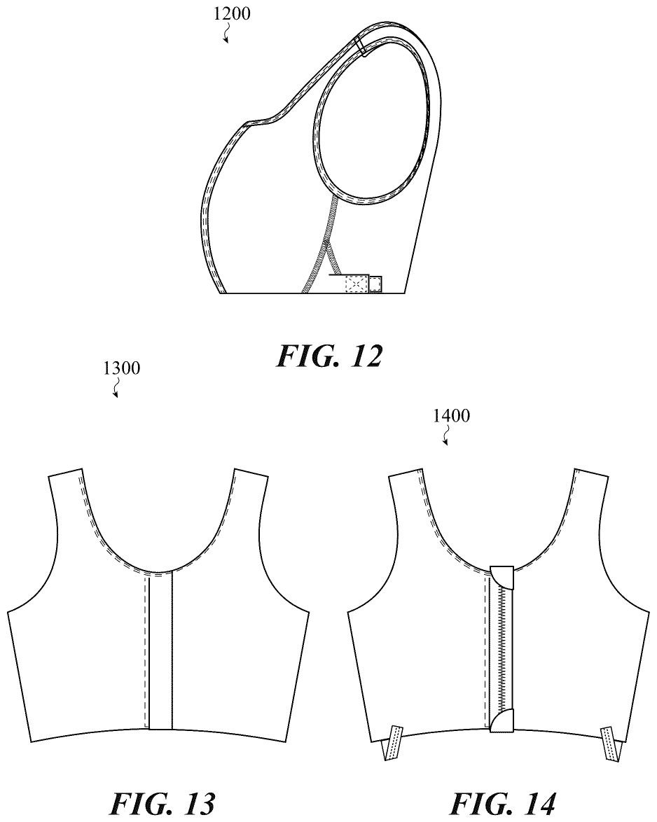

FIG. 12 illustrates a side view of a garment 1200. The garment 1200 includes a layered structure having a first fabric layer and a second fabric layer, which may be referred to as an interior layer and an exterior layer. Each layer may exhibit different properties, including elasticity, thermal properties, moisture wicking properties, and air permeability properties. A thermoplastic polyurethane bonding may be positioned between the first fabric layer and the second fabric layer. The thermoplastic polyurethane bonding secures the first fabric layer to the second fabric layer along a contour of the garment 1200.

FIG. 13 illustrates a front view of a garment 1300. The garment 1300 includes a torso-covering body with shoulder straps and a front opening region. The front opening region of the garment 1300 includes a seam extending vertically along a central portion of the torso-covering body. The seam may be covered by a fabric flap such that the seam itself does not contact or irritate skin surfaces.

FIG. 14 illustrates a rear view of a garment 1400. The garment 1400 includes a torso-covering body with shoulder straps and a rear opening region. The rear opening region includes a fastening mechanism (e.g., a zipper, clasp, magnetic teeth engagement, or the like) disposed vertically along a central portion of the torso-covering body. The fastening mechanism of the garment 1400 includes a securing element positioned to maintain closure of the garment 1400 during use.

These foregoing embodiments depicted in FIG. 1-14 and the various alternatives thereof and variations thereto are presented, generally, for purposes of explanation, and to facilitate an understanding of various configurations and constructions of a garment, such as described herein. However, it will be apparent to one skilled in the art that some of the specific details presented herein may not be required in order to practice a particular described embodiment, or an equivalent thereof.

Thus, it is understood that the foregoing and following descriptions of specific embodiments are presented for the limited purposes of illustration and description. These descriptions are not targeted to be exhaustive or to limit the disclosure to the precise forms recited herein. To the contrary, it will be apparent to one of ordinary skill in the art that many modifications and variations are possible in view of the above teachings.

For example, garments described herein may be understood as generally directed toward post-surgical recovery applications in which extended wear of a torso-covering body is required. The garment construction includes a first fabric layer and a second fabric layer that are secured together through a thermoplastic polyurethane bonding. The thermoplastic polyurethane bonding can extend between the fabric layers in order to maintain positional stability of the torso-covering body without introducing rigid components against the skin. The layered bonding arrangement provides structural integrity of the garment while maintaining flexibility across torso regions.

The garment structure may include a pair of shoulder straps extending upwardly from the torso-covering body in order to retain the garment in a proper position on the torso. The garment construction may be configured with both front and rear opening regions. In some embodiments, the front opening region is defined by a vertically oriented seam disposed centrally across the torso-covering body. In other embodiments, the rear opening region includes a fastening mechanism extending along a vertical axis of the garment body. The fastening mechanism can include a securing element arranged to maintain closure of the garment during wear.

The garment construction can incorporate elastic properties into one or more fabric layers in order to provide a balanced degree of stretch across the torso-covering body. The layered construction may further leverage bonded reinforcement in regions adjacent to seams, arm openings, and strap junctions in order to stabilize stress concentrations during wear.

Additional embodiments may include fastening mechanisms such as hook-and-loop strips, zippers, clasps, or snap structures. The fastening mechanisms can be disposed along vertical closure lines in either the front or rear regions. The fastening elements may be bonded or stitched to the fabric layers in a manner that maintains a flat interior surface along the skin-contacting regions.

Broadly, the garments described herein provide a bonded multilayer construction in which fabric layers may be joined through thermoplastic polyurethane bonding. The garments further provide variations in seam placement, fastening mechanisms, and strap configurations.

The layered bonded arrangement may be leveraged for post-surgical garments intended for extended wear, with construction methods relying upon bonded fabric layering rather than rigid inserts or external reinforcing components.

As used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

One may appreciate that although many embodiments are disclosed above, that the operations and steps presented with respect to methods and techniques described herein are meant as exemplary and accordingly are not exhaustive. One may further appreciate that alternate step order or fewer or additional operations may be required or desired for particular embodiments.

Although the disclosure above is described in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the some embodiments of the invention, whether or not such embodiments are described and whether or not such features are presented as being a part of a described embodiment. Thus, the breadth and scope of the present invention should not be limited by any of the above-described exemplary embodiments but is instead defined by the claims herein presented.

What is claimed is:

1. A garment comprising:
a torso covering body formed from a fabric comprising 90 percent nylon and 10 percent elastane;
a first fabric layer and a second fabric layer joined by a thermoplastic polyurethane bonding;
a pair of shoulder straps extending from a neckline region toward an underarm region;
a band disposed along a lower edge of the torso covering body;
a front closure disposed along a central portion of the torso covering body; and
a plurality of drainage tube support straps positioned on the torso covering body and configured to retain a drainage conduit adjacent to the torso covering body.

2. The garment of claim 1, wherein the drainage tube support straps are formed from elastic fabric.

3. The garment of claim 1, wherein the drainage tube support straps are disposed adjacent to the band.

4. The garment of claim 1, wherein the drainage tube support straps are arranged symmetrically on left and right sides of the torso covering body.

5. The garment of claim 1, wherein each drainage tube support strap defines a loop sized to receive the drainage conduit.

6. The garment of claim 1, wherein the drainage conduit extends through an outlet port disposed in the band.

7. The garment of claim 6, wherein the drainage conduit is coupled to a collection reservoir or pump positioned externally of the torso covering body.

8. The garment of claim 1, wherein the front closure comprises a zipper.

9. The garment of claim 1, wherein the front closure comprises a hook-and-loop fastener.

10. A garment comprising:
a torso covering body comprising at least 80 percent nylon and at least 10 percent elastane;
a first fabric layer and a second fabric layer bonded together by a thermoplastic polyurethane layer;
a pair of shoulder straps extending from a neckline region toward an underarm region, the shoulder straps being configured at least in part to be cut by a wearer to adjust a size and level of support of the torso covering body;
a band extending along a lower edge of the torso covering body; and
a closure disposed along a central portion of the torso covering body.

11. The garment of claim 10, wherein the closure comprises a zipper extending vertically along the central portion of the torso covering body.

12. The garment of claim 10, wherein the band includes a plurality of securing elements positioned to tighten the torso covering body around the wearer.

13. A garment comprising:
a torso covering body constructed from a fabric including 90 percent nylon and 10 percent elastane;
a first fabric layer and a second fabric layer bonded together by a thermoplastic polyurethane layer;
a pair of shoulder straps extending from a neckline region toward an underarm region;
a closure disposed along a front central portion of the torso covering body;
a pair of side panels positioned between a front panel and a back panel of the torso covering body, the side panels being formed from a material different from a material of the front panel and the back panel; and
a plurality of seams joining the panels of the torso covering body, the seams being covered by fabric such that no plastic layer or stitching is exposed to a skin-contacting surface of the torso covering body.

14. The garment of claim 13, wherein the side panels are constructed from mesh fabric.

15. The garment of claim 13, wherein the seams are covered by folded extensions of the first fabric layer and second fabric layer.

16. The garment of claim 13, wherein the closure comprises a zipper extending vertically along the front central portion of the torso covering body.

17. The garment of claim 13, wherein the closure comprises hook-and-loop fasteners extending along the front central portion of the torso covering body.

18. The garment of claim 1, wherein the drainage tube support straps are cuttable by a wearer to permit removal from the torso covering body.

19. The garment of claim 10, wherein the shoulder straps extend continuously from the neckline region to a back panel of the torso covering body.

20. The garment of claim 13, wherein the side panels are disposed laterally between the front closure and the back panel of the torso covering body.
